(19) United States
(12) Patent Application Publication
(10) Pub. No.: US 2024/0066143 A1
(43) Pub. Date: Feb. 29, 2024

(54) NATURAL-POLYPHENOLS-BASED MULTI-STAGE POROUS HYDROGEL SUSTAINED RELEASE DRUG DELIVERY SYSTEM AND ITS PREPARATION METHOD

(71) Applicant: Sichuan University, Chengdu (CN)

(72) Inventors: Junling GUO, Chengdu (CN); Jiaojiao SHANG, Chengdu (CN); Jiezhou PAN, Chengdu (CN)

(73) Assignee: Sichuan University, Chengdu (CN)

(21) Appl. No.: 18/346,290

(22) Filed: Jul. 3, 2023

Related U.S. Application Data
(63) Continuation of application No. PCT/CN2022/119186, filed on Sep. 16, 2022.

(30) Foreign Application Priority Data
Aug. 31, 2022 (CN) 2022110560038

(51) Int. Cl. A61K 47/69; A61K 36/185; A61K 47/02; A61K 47/26

(52) U.S. Cl. CPC A61K 47/6903; A61K 36/185; A61K 47/02; A61K 47/26

(57) ABSTRACT

This invention discloses a natural polyphenols-based, multi-stage porous hydrogel sustained release drug delivery system, which relates to the field of drug delivery. It consists of the hydrogel matrix and the supramolecular filler, which is a complex of natural bio-based polymers and metal ions. The natural bio-based polymer can be natural polyphenols, dopamine or its derivatives, polysaccharide biomass, or protein biomass. The hydrogel matrix can be chitosan, carboxymethyl chitosan, sodium alginate, carboxymethyl cellulose, hyaluronic acid, collagen, gelatin, or agarose. The metal ion can be one of the cations of Al, Fe, Zn, Mn, Ni, Co, or V. By this invention, the supramolecular filler based on natural bio-based polymer can form in situ in the hydrogel, to regulate the pores of the hydrogel and interact with different drugs, thus adjusting the drug release rate.

### CROSS-REFERENCE TO RELATED APPLICATIONS

The application claims priority to Chinese patent application No. 2022110560038, filed on Aug. 31, 2022, the entire contents of which are incorporated herein by reference.

### TECHNICAL FIELD

This invention relates to the field of drug delivery technology, in particular to a natural polyphenols-based, multi-stage porous hydrogel sustained release drug delivery system, and its preparation method.

### BACKGROUND

Hydrogels are a highly desirable drug delivery system that finds applications in various fields of medicine, such as cardiology, oncology, immunology, and wound healing. It is a polymer material formed through physical or chemical crosslinking, resulting in a three-dimensional reticulate structure. By using water as the dispersion medium, hydrogels can swell by absorbing a large amount of water while maintaining their structural stability. The high water content (typically 70-99%) provides them with physical properties similar to biological tissues, and gives them excellent biocompatibility as well as the ability to encapsulate hydrophilic drugs. In addition, as hydrogels are usually prepared in aqueous solutions, the risk of denaturation and aggregation for the drugs to be exposed to organic solvents is minimized. The cross-linked polymer network gives hydrogels a solid appearance, as well as adjustable mechanical properties. For example, the stiffness of hydrogels can be adjusted from 0.5 KPa to 5 MPa to match the soft tissues in different parts of the human body.

However, as the pore size of hydrogels is much larger than the hydro-mechanical volume of drug molecules, drug release through hydrogels often results in burst release. Releasing the drug too quickly or too early can result in the accumulation of excessive drug concentration in a short period of time, which may lead to a series of side effects and affect the ultimate efficacy of the drug. By adjusting the structure of hydrogels, such as modification of the internal network of hydrogels, or regulating their pore structure, we can make specific hydrogel structures control the release of certain kinds of drugs. However, this highly specific structural design makes the clinical design and transformation of hydrogels costly. Therefore, it is promising to build a new universal hydrogel drug delivery system that enables the continuous and stable release of different kinds of drugs.

### SUMMARY

Due to the high costs incurred in the clinical design and transformation of existing hydrogel modification methods, this invention provides a universal, natural polyphenols-based multi-stage porous hydrogel sustained release drug delivery system, along with its preparation method, to achieve the continuous and stable release of different kinds of drugs.

The natural polyphenols-based, multi-stage porous hydrogel sustained release drug delivery system provided by this invention consists of the hydrogel matrix and the supramolecular filler. The said supramolecular filler is a complex of natural bio-based polymer and metal ions. The said natural bio-based polymer can be one of natural polyphenols, dopamine or its derivatives, polysaccharide biomass, or protein biomass. Drugs that can be used for controlled release with this system include small molecule drugs, peptide drugs, protein drugs, and nucleic drugs.

The said hydrogel matrix can be chitosan, carboxymethyl chitosan, sodium alginate, carboxymethyl cellulose, hyaluronic acid, collagen, gelatin, or agarose.

The said natural polyphenol can be bayberry tannin, persimmon tannin, black wattle bark tannin, larch tannin, tannic acid, ellagic acid, epigallocatechin gallate, catechin gallate, anthocyanin, or catechin. The said polysaccharide biomass can be chitosan, carboxymethyl chitosan, cellulose, carboxymethyl cellulose, or hyaluronic acid. The said protein biomass can be gelatin or collagen.

The said metal ion can be one of the cations of Al, Fe, Zn, Mn, Ni, Co or V, with good biocompatibility.

A preferable weight ratio of supramolecular filler to hydrogel matrix is 1:(5-10).

The drugs suitable for the natural polyphenols-based, multi-stage porous hydrogel sustained release drug delivery system of this invention include lidocaine, verapamil, terazosin, doxorubicin, vancomycin, insulin, aptamer AS1411, interleukin-6, and interleukin-10.

The hydrogel sustained release drug delivery system of this invention consists of the hydrogel matrix based on natural biomass, and the supramolecular filler based on a natural bio-based polymer for regulating the pores of the hydrogel and interacting with different drugs. These interactions are mainly achieved through the multiple interactions between the phenolic hydroxyl groups on the supramolecular fillers based on natural bio-based polymers and the hydrogel's molecular chains, including hydrophilic interaction, hydrogen bonding, π-π stacking, electrostatic interaction, metal complexation, etc.

The steps for preparing the natural polyphenols-based, multi-stage porous hydrogel sustained release drug delivery system are as follows:

S1: Fully mix the aqueous solution of a metal salt with the natural bio-based polymer solution, and adjust the pH value to 7.0 for complete reaction, to obtain the supramolecular filler.

S2: Dissolve the hydrogel matrix in deionized water, add the supramolecular filler, stir evenly, add the drug, and stir thoroughly for full mixing. The said drug can be lidocaine, verapamil, terazosin, doxorubicin, vancomycin, insulin, aptamer AS1411, interleukin-6, or interleukin-10.

S3: Add the crosslinker into the solution obtained in step S2, stir vigorously and evenly, and let stand for crosslinking, to get the gel. The said crosslinker can be calcium chloride, glutaraldehyde, genipin, or bissulfosuccinimidyl suberate, or a compound of calcium carbonate and gluconolactone.

Preferably, in step S1, adjust the pH to 7.0 with aqueous sodium hydroxide solution or PBS buffer.

Preferably, step S2 should be as follows: add the hydrogel matrix into deionized water, heat to 50° C., stir until the hydrogel matrix dissolves, add the supramolecular filler, stir evenly, cool down to 25° C., and add the drug.

Compared with the prior art, this invention is beneficial in that:

(1) The supramolecular filler based on natural bio-based polymer will form in situ in the hydrogel to regulate the pores of the hydrogel and interact with different drugs, thus adjusting the release rate of the drugs. Specifically, multiple interactions, such as π-π interaction, hydrogen bonding, electrostatic interaction, hydrophilic interaction, and metal complexation, will form between the supramolecular filler and the drug molecules, thus, through the chemical bonding, the release rate of drug molecules will be reduced and the effect of drug release control achieved. As a material made of natural biomass, the supramolecular filler is low-cost and has no side effects.

(2) The supramolecular filler based on natural bio-based polymer provided by this invention can form in the hydrogel a multi-scale porous structure, including the micro-porous structure of the filler itself, the meso-porous structure between the filler nanoparticles, and the macro-porous structure between the filler and the hydrogel's network structure. The multi-scale porous structure provides corresponding diffusion channels for drug molecules of different molecular weights, and therefore, the release of drug systems of different molecular weights can be controlled simultaneously.

(3) The multi-scale porous structure formed according to this invention in the hydrogel structure by supramolecular filler based on natural bio-based polymer can greatly increase the sinuosity and complexity of the hydrogel's internal network, thus improving the diffusion paths required by drug molecules that diffuse in the hydrogel and thus controlling the release rate of these molecules.

(4) The natural biomass-based hydrogel sustained release drug delivery system prepared according to this invention can completely avoid the burst release of the drugs loaded therein in the first 24 h, with an average daily-accumulated drug release of less than 10%.

(5) This invention has the advantages of simple preparation and low-cost industrial production. The materials used are natural, bio-based and of high safety levels, and the hydrogel produced can maintain stable structure in transportation and storage, suitable for use in the field of drug delivery.

Other advantages, purposes and characteristics of this invention will be reflected partly in the following descriptions, and partly will be understood by those working in the field through study and practice of this invention.

### BRIEF DESCRIPTION OF DRAWINGS

FIG. 1. TEM image of the supramolecular filler based on natural bio-based polymer prepared in Embodiment 1.

FIG. 2. Pore size distribution diagram of the supramolecular filler based on natural bio-based polymer prepared in Embodiment 1.

FIG. 3. Drug release experimental results of the hydrogels prepared respectively in Embodiment 1 and in Control 1.

FIG. 4. Drug release experimental results of the hydrogels prepared respectively in Embodiment 2 and in Control 2.

FIG. 5. Pore size analysis diagram of the drug releasing hydrogel based on natural biomass prepared in Embodiment 3.

FIG. 6. Drug release experimental results of the hydrogels prepared respectively in Embodiment 3 and in Control 3.

FIG. 7. Comparison of scanning electron microscope images of the hydrogels prepared respectively in Embodiment 4 and in Control 4.

FIG. 8. Drug release experimental results of the hydrogels prepared respectively in Embodiment 4 and in Control 4.

### DETAILED DESCRIPTION OF THE EMBODIMENTS

The preferred embodiments of this invention will be illustrated in combination with the attached drawings as follows, and it should be understood that the preferred embodiments described herein are only for the purpose of illustration and explanation of this invention, not for limitation of this invention.

### Embodiment 1

The method for preparing the natural polyphenols-based, multi-stage porous hydrogel sustained release drug delivery system is as follows:

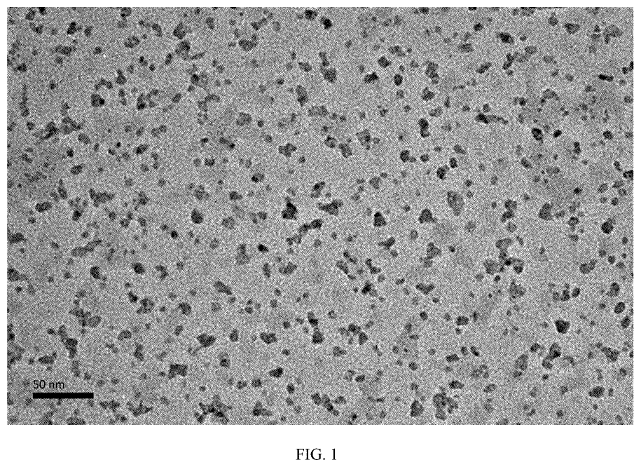

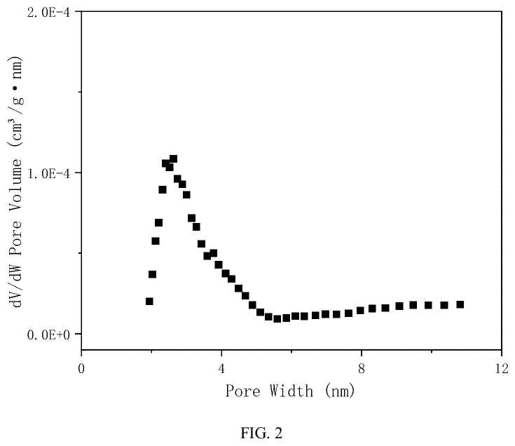

At 30° C., mix 1 mL of tannic acid (40 mg/mL) with 1 mL of ferric sulfate hexahydrate solution (10 mg/mL) evenly, let stand for 10 min, and add 50 μL of sodium hydroxide solution (1 mol/L), to complete the self-assembly of the complexes of tannic acid and iron ions. After centrifuging the solution at 10,000 r/min, freeze-dry the precipitation to obtain the supramolecular filler based on natural bio-based polymers. Use a TEM to observe its morphology and use a BET to test its pore sizes. The test results are shown in FIG. 1 and FIG. 2. As can be seen from FIGS. 1 and 2, the supramolecular filler based on natural bio-based polymers and formed with tannic acid and iron ions has a particle size of about 20 nm, and a pore size of 2-8 nm, which is suitable for the diffusion of small molecule drugs. After that, add 5 g of sodium alginate and then 100 g of deionized water into a three-neck flask, keep stirring and heat to 50° C., and stir for 30 minutes until the sodium alginate dissolves. Add 1 g of the supramolecular filler based on natural bio-based polymer, stir evenly and cool down to 25° C. Add 0.1 g of anhydrous calcium carbonate, stir evenly, and add into the reaction vessel 0.5 g of the drug, which is lidocaine hydrochloride in this case. Add 0.5 g of gluconolactone into the product, stir vigorously, transfer the mixture into a mold, and let stand for crosslinking. Finally, demold the shaped bulk gel to obtain the natural biomass-based, drug-releasing hydrogel.

Control 1: Based on Embodiment 1, the hydrogel as a control sample was prepared without doping supramolecular fillers based on natural bi-based polymers, for the release of Novocaine hydrochloride. Preparation of the control sample:

Add 5 g of sodium alginate and then 100 g of deionized water into a three-neck flask, keep stirring and heat to 50° C. Stir for 30 minutes until the sodium alginate dissolves, cool down to 25° C. Add 0.1 g of anhydrous calcium carbonate, stir evenly, and add into the reaction vessel 0.5 g of drug, which is lidocaine hydrochloride in this case. Add 0.5 g of gluconolactone into the product, stir vigorously, transfer the mixture into the mold, and let stand for crosslinking. Finally, demold the shaped bulk gel to obtain the conventional hydrogel as the control sample.

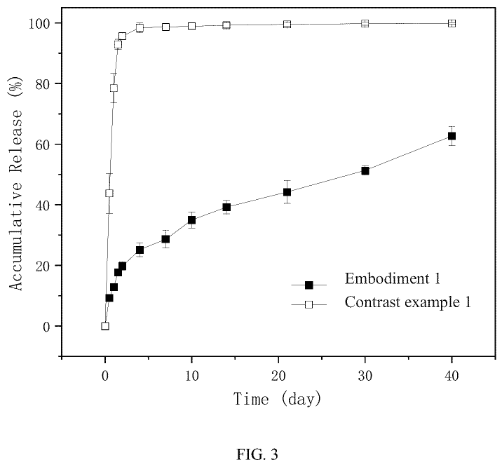

10 g of the hydrogels prepared in Embodiment 1 and in Control 1 were placed into 500 mL of PBS buffers respectively, and 1 mL of the supernatant was taken every day to check the drug release by an HPLC. The results are as shown in FIG. 3. The experimental results prove that the natural biomass-based drug releasing hydrogel that was prepared according to this invention can effectively sustain the release of small molecule hydrophilic drugs.

### Embodiment 2

The method for preparing the natural polyphenols-based, multi-stage porous hydrogel sustained release drug delivery system is as follows:

At 30° C., mix 1 mL of tannic acid (40 mg/mL) with 1 mL of aluminum chloride solution (10 mg/mL) evenly, let stand for 10 min, and add 50 μL of sodium hydroxide solution (1 mol/L), to complete the self-assembly of the complexes of tannic acid and aluminum ions. After centrifuging the solution at 10,000 r/min, freeze-dry the precipitation to obtain the supramolecular filler based on natural bio-based polymers. After that, add 10 g of gelatin and then 100 g of deionized water into a three-neck flask, keep stirring and heat to 50° C. Stir for 30 minutes until the gelatin dissolves, and add 1 g of supramolecular filling based on natural bio-based polymer. Stir evenly, cool down to 25° C., and add into the reaction vessel 0.5 g of the drug, which is terazosin hydrochloride in this case. Add 0.2 g of genipin into the product, stir vigorously, transfer the mixture into a mold, and let stand for crosslinking. Finally, demold the shaped bulk gel to obtain the drug releasing hydrogel based on natural biomass.

Control 2: Based on Embodiment 2, the hydrogel as a control sample was prepared without doping supramolecular fillers based on natural bio-based polymers, for the release of terazosin hydrochloride. Preparation of the control sample:

Add 10 g of gelatin and then 100 g of deionized water into a three-neck flask, keep stirring and heat to 50° C. Stir for 30 minutes until the gelatin dissolves. Cool down to 25° C., and add into the reaction vessel 0.5 g of the drug, which is terazosin hydrochloride in this case; add 0.2 g of genipin into the product, stir vigorously, transfer the mixture into a mold, and let stand for crosslinking; and finally, demold the shaped bulk gel to obtain the conventional hydrogel as a control sample.

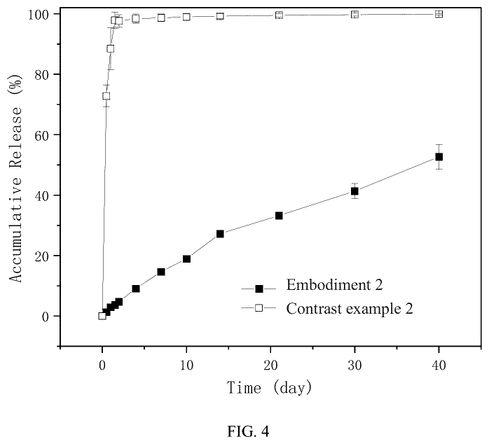

10 g of the hydrogels prepared in Embodiment 2 and in Control 2 were placed into 500 mL of PBS buffers respectively, and 1 mL of the supernatant was taken every day to check the drug release by an HPLC. The results are as shown in FIG. 4. The experimental results prove that the natural biomass-based drug releasing hydrogel that was prepared according to this invention can effectively sustain the release of small molecule hydrophilic drugs.

### Embodiment 3

The method for preparing the natural polyphenols-based, multi-stage porous hydrogel sustained release drug delivery system is as follows:

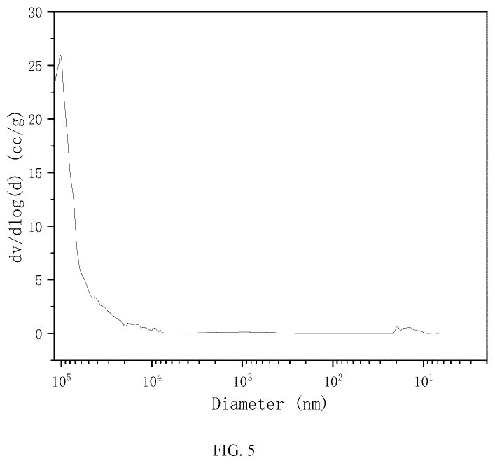

At 30° C., mix 1 mL of catechin gallate (20 mg/mL) with 1 mL of ferric sulfate hexahydrate (10 mg/mL) evenly, let stand for 10 min, and add 20 μL of sodium hydroxide solution (1 mol/L), to complete the self-assembly of the complexes of tannic acid and aluminum ions. After centrifuging the solution at 10,000 r/min, freeze-dry the precipitation to obtain the supramolecular filler based on natural bio-based polymers. After that, add 8 g of carboxymethyl chitosan and then 100 g of deionized water into a three-neck flask. Keep stirring and heat to 50° C., and stir for 30 minutes until the carboxymethyl chitosan dissolves. Add 1 g of supramolecular filler based on natural bio-based polymer, stir evenly, cool down to 25° C., and add 0.5 g of the drug into the reaction vessel, which is irinotecan hydrochloride in this case. Add 0.2 g of genipin into the product, stir vigorously, transfer the mixture into a mold, and let stand for crosslinking. Finally, demold the shaped bulk gel to obtain the drug-releasing hydrogel based on natural biomass. A sample of the drug releasing hydrogel based on natural biomass was taken, freeze-dried, and tested with an automatic mercury injection instrument for pore size analysis. The results are as shown in FIG. 5. As can be seen from FIG. 5, in the hydrogel there are pores of 10 nm-20 nm, which are the pores between the molecules of the supramolecular filler based on natural bio-based polymers, in line with the multiple porosity theory of this invention about the drug releasing hydrogel based on natural biomass.

Control 3: Based on Embodiment 3, a hydrogel as a control sample was prepared without doping supramolecular fillers based on natural bio-based polymers, for the release of irinotecan hydrochloride. Preparation of the control sample:

Add 8 g of carboxymethyl chitosan and then 100 g of deionized water into a three-neck flask. Keep stirring, heat to 50° C., and stir for 30 minutes until carboxymethyl chitosan dissolves. Cool down to 25° C., and add into the reaction vessel 0.5 g of the drug, which is irinotecan hydrochloride in this case. Add 0.2 g of genipin into the product, stir vigorously, transfer the mixture into a mold, and let stand for crosslinking. Finally, demold the shaped bulk gel to obtain the drug-releasing hydrogel based on natural biomass.

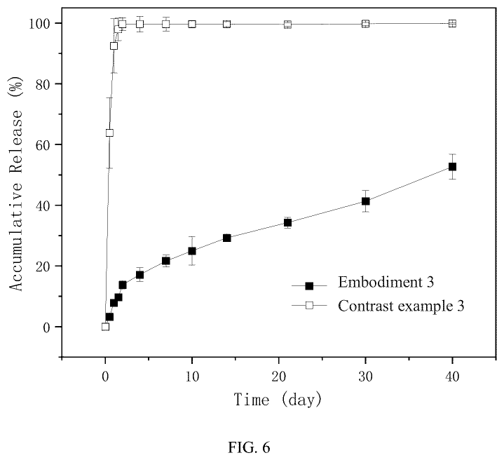

10 g of the hydrogels prepared in Embodiment 3 and Control 3 were placed into 500 mL PBS buffers respectively, and 1 mL of the supernatant was taken every day to check the drug release by a HPLC. The results are as shown in FIG. 6.

### Embodiment 4

The method for preparing the natural polyphenols-based, multi-stage porous hydrogel sustained release drug delivery system is as follows:

At 30° C., mix 1 mL of black wattle bark tannin (40 mg/mL) with 1 mL of zinc chloride solution (10 mg/mL) evenly, let stand for 10 min, and add 20 μL of sodium hydroxide solution (1 mol/L), to complete the self-assembly of the complexes of black wattle bark tannin and zinc ions. After centrifuging the solution at 10,000 r/min, freeze-dry the precipitation to obtain the supramolecular filler based on natural bio-based polymers. After that, add 8 g of carboxymethyl chitosan and then 100 g of deionized water into a three-neck flask. Keep stirring, heat to 50° C., stir for 30 minutes until the sodium alginate dissolves. Add 1 g of the supramolecular filler based on natural bio-based polymers, stir evenly, cool down to 25° C., and add into the reaction vessel 0.5 g of drug, which is verapamil in this case. Add 0.2 g of genipin into the product, stir vigorously, transfer the mixture into the mold, and let stand for crosslinking. Finally, demold the shaped bulk gel to obtain the drug-releasing hydrogel based on natural biomass, and freeze-dry it.

Control 4: Based on Embodiment 4, and with the same method, a sample of conventional hydrogel was prepared without adding supramolecular fillers based on natural bio-based polymers. Preparation: add 8 g of carboxymethyl chitosan and then 100 g of deionized water into a three-neck flask. Keep stirring and heat to 50° C., stir for 30 minutes until sodium alginate dissolves, and cool down to 25° C. Add into the reaction vessel 0.5 g of the drug, which is verapamil in this case. Add 0.2 g of genipin into the product, stir vigorously, transfer the mixture into a mold, and let stand for crosslinking. Finally, demold the shaped bulk gel to obtain the conventional drug releasing hydrogel, and freeze-dry it.

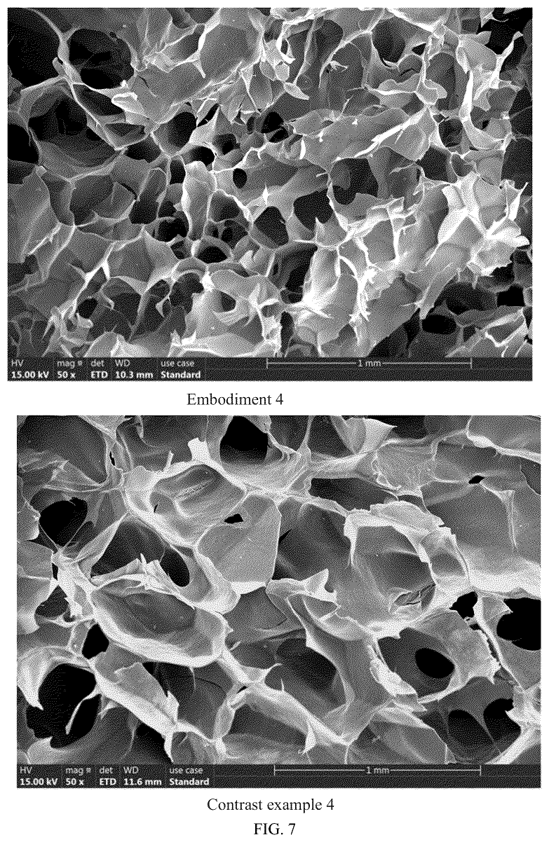

The pore sizes of the hydrogels prepared in Embodiment 4 and in Control 4 were analyzed by a scanning electron microscope, and the results are as shown in FIG. 7. As can be seen from FIG. 7, the pores in hydrogel shrunk obviously after supramolecular fillers based on natural bio-based polymers were added, which proves the regulation effect of supramolecular fillers based on natural bio-based polymers on the pores of hydrogels.

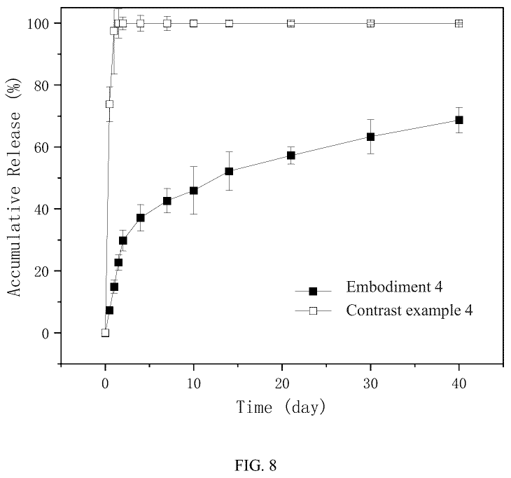

10 g of hydrogels prepared in Embodiment 4 and of in Control 4 were respectively placed into 500 mL of PBS buffer, and 1 mL of supernatant was taken every day to check the drug release by an HPLC. The results are as shown in FIG. 8.

The above are only comparatively good embodiments of this invention, not intended to limit this invention in any form. Although this invention has been disclosed as above with comparatively good embodiments, they are not intended to restrict this invention. Any technical person familiar with the profession, within the scope of the technical scheme of this invention, can make use of the technical contents disclosed herein, and can change or modify them to some extent into equivalent embodiments or variants. However, any simple change, variation or modification to the embodiments mentioned above, which is in line with the technical essence of this invention and is not independent of the contents of the technical scheme of this invention, shall still fall within the scope of the technical scheme of this invention.

What is claimed is:

1. A natural polyphenols-based, multi-stage porous hydrogel sustained release drug delivery system, characterized in that it comprises the hydrogel matrix and the supramolecular filler which is a complex of natural biological based polymer and metal ions, wherein the natural bio-based polymer can be natural polyphenols, dopamine and its derivatives, polysaccharide biomass, or protein biomass.

2. The natural polyphenols-based, multi-stage porous hydrogel sustained release drug delivery system as described in claim 1 is characterized in that the hydrogel matrix can be chitosan, carboxymethyl chitosan, sodium alginate, carboxymethyl cellulose, hyaluronic acid, collagen, gelatin, or agarose.

3. The natural polyphenols-based multi-stage porous hydrogel sustained release drug delivery system as described in claim 1 is characterized in that the metal ion can be one of the cations of Al, Fe, Zn, Mn, Ni, Co, or V.

4. The natural polyphenols-based, multi-stage porous hydrogel sustained release drug delivery system as described in claim 1 is characterized in that the natural polyphenol can be bayberry tannin, persimmon tannin, black wattle bark tannin, larch tannin, tannin, ellagic acid, epigallocatechin gallate, catechin gallate, anthocyanin, or catechin.

5. The natural polyphenols-based, multi-stage porous hydrogel sustained release drug delivery system as described in claim 1 is characterized in that the polysaccharide biomass can be chitosan, carboxymethyl chitosan, cellulose, carboxymethyl cellulose, or hyaluronic acid.

6. The natural polyphenols-based, multi-stage porous hydrogel sustained release drug delivery system as described in claim 1 is characterized in that the protein biomass can be gelatin or collagen.

7. The natural polyphenols-based, multi-stage porous hydrogel sustained release drug delivery system as described in claim 1 is characterized by a weight ratio of supramolecular fillers to hydrogel matrix of 1:(5-10).

8. A method for preparing the natural polyphenols-based, multi-stage porous hydrogel sustained release drug delivery system as described in claim 1 is characterized by the following steps:
S1. Mix the aqueous solution of metal salt with the natural bio-based polymer solution, and adjust the pH value to 7.0 for a complete reaction, to obtain the supramolecular filler;
S2. Dissolve the hydrogel matrix in deionized water, add supramolecular fillers, stir evenly, add the drug, and stir thoroughly for full mixing;
S3: Add the crosslinker into the solution obtained in step S2, stir vigorously and evenly, and let stand for crosslinking, to get the gel.

9. The method for preparing the natural biomass-based hydrogel sustained release drug delivery system as described in claim 8 is characterized by step S2, specifically: Add hydrogel matrix into deionized water, heat to 50° C., stir until the hydrogel matrix is dissolved, add supramolecular filler, stir evenly, cool down to 25° C., and add the drug.

10. The method for preparing the natural polyphenols-based, multi-stage porous hydrogel sustained release drug delivery system as described in claim 8 is characterized in that the said crosslinker can be calcium chloride, glutaraldehyde, genipin, or bissulfosuccinimidyl suberate.
